(19) United States
(12) Patent Application Publication
(10) Pub. No.: US 2022/0033907 A1
(43) Pub. Date: Feb. 3, 2022

(54) BIOMARKERS

(71) Applicants: University College Dublin, National University of Ireland Dublin, Bettietd,Dublin (IE); Royal College of Surgeons in Ireland, Dublin (IE)

(72) Inventors: Chris Watson, Dublin (IE); Mark Ledwidge, Dublin (IE); John Baugh, Dublin (IE); Nadezhda Glezeva, Dublin (IE); Sudipto Das, Dublin (IE)

(21) Appl. No.: 17/430,877

(22) PCT Filed: Feb. 14, 2020
(86) PCT No.: PCT/EP2020/053937

§ 371 Date: Aug. 13, 2021

(30) Foreign Application Priority Data
Feb. 14, 2019 (GB) 1902077.5

(51) Int. Cl. C12Q 1/6883; G16H 50/30; G16H 50/20

(52) U.S. Cl. CPC C12Q 1/6883; G16H 50/30; C12Q 2600/178; C12Q 2600/154; C12Q 2600/158; G16H 50/20

(57) ABSTRACT

The invention provides a method of prognosing and/or diagnosing heart disease or heart failure in a subject, comprising determining the methylation status and/or expression level of at least one methylation marker selected from the group consisting of MFSD2B, miR24-1, TTPA, GALNT15, ITGBL1, SMOC2, MSR1, PVT1, MYOM3, COX17, MYBPC3, HEY2, and MRPL44 wherein the methylation status and/or expression level of at least one methylation marker is indicative of the prognosis and/or diagnosis of said subject. A panel of biomarkers, means, a kit and a device for use in assessing risk of HCM, ISCM and DCM are disclosed.

The present invention relates to biomarkers and in particular panels of methylation biomarkers and their use in prognosing, diagnosing and/or treatment of heart disease and heart failure.

### BACKGROUND

Heart failure (HF) is a major public health problem which affects approximately 2% of the world's population, extending to more than 10% in the over 65 year-old group^{1,2}. With projections showing that the prevalence of HF will increase by 46% from 2012 to 2030³, it is imperative to find more effective means to screen and diagnose cardiac insufficiency in its early phase. Efforts to do so must take into account the multiple etiologies and facets that make up the complexity of the HF syndrome. Some of the leading causes for HF include chronic hypertension causing left ventricular hypertrophy with concentric, at first, and later eccentric cardiac remodeling; subclinical atherosclerosis and peripheral vascular disease; ischemic heart disease causing myocardial infarction (MI); and cardiomyopathies, including hypertrophic (HCM), dilated (DCM), arrhythmogenic right ventricular cardiomyopathy, and acquired—ischemic cardiomyopathy (ISCM) and myocarditis.

The causes and events driving the progression of these disorders which predispose to HF and contribute to the different HF pathophysiologies have not been fully unveiled. Mounting evidence from studies over the past years has come to depict a multifaceted schematic suggesting a role for genetic factors, environmental stimuli, and lifestyle choices that ultimately contribute to the course of events culminating in HF. This process is known as pathological cardiac remodeling and is phenotypically characterized by adverse changes in the size, shape, and structure of the heart. At the molecular level, these aberrant phenotypic changes and traits are controlled by a complex genetic network which when perturbed, potentially results in generation of aberrant gene expression patterns within heart tissue. Mechanisms which potentially regulate gene expression in the heart have thus gained importance and efforts are thus being currently made to elucidate the precise pathways and molecules which can be targeted pharmacologically in order to ameliorate adverse cardiac remodeling and HF. One such crucial mechanism regulating gene expression involves epigenetic modifications such as DNA methylation, covalent histone modifications, ATP-dependent chromatin remodeling, and non-coding RNAs, including micro RNA (miRNA) and long non-coding RNA (IncRNA). Several comprehensive reports have suggested their plausible role in HF pathogenesis⁴⁻⁷. Specifically, DNA methylation is a unique physiological process for fine-tuning of gene expression in line with the needs of the body and in response to the ever-changing environmental milieu⁸. It occurs when a methyl group is added to the 5′ position of the cytosine ring within CpG sites or islands in the DNA to create 5-methylcytosine. This process is conserved and is commonly linked to transcriptional gene repression as it can prevent binding of transcription factors to the DNA or limit the access to gene regulatory regions.

Aberrant patterns of DNA methylation have been shown to contribute to maladaptive cardiac remodelling including hypertrophy, fibrosis, ischemia, and inflammation⁹. To date, studies that have performed DNA methylation profiling in HF patients have used whole-genome bisulfite sequencing techniques to assess global changes in methylation and epigenomic patterns in blood or cardiac tissue from patients from a single HF aetiology (end-stage ischemic/idiopathic HF¹⁰, DCM¹¹⁻¹⁴, ISCM^{15, 16}) compared to a non-HF control group. Novel genes whose expression is controlled by DNA methylation have been identified in DCM¹¹⁻¹³ and ISCM^{15, 16}. However, all these methylation studies have been limited to the study of a single HF patient cohort and moreover none of them have examined DNA methylation signatures in other significant HF aetiologies such as HCM, in particular obstructive HCM (HOCM). Such methylation signatures could be used to discover novel diagnostic and therapeutic targets for this incurable disease.

### SUMMARY OF THE INVENTION

The invention provides a method of prognosing and/or diagnosing heart disease or heart failure in a subject, comprising

determining the methylation status and/or expression level of at least one methylation marker selected from the group consisting of MFSD2B, miR24-1, TTPA, GALNT15, ITGBL1, SMOC2, MSR1, PVT1, MYOM3, HEY2 and MRPL44

wherein the methylation status and/or expression level of at least one methylation marker is indicative of the prognosis and/or diagnosis of said subject.

Alternatively or in addition, the at least one methylation marker is selected from the group consisting of COX17 or MYBPC3.

The method can be carried out on a sample from a patient.

The sample can be blood, cardiac tissue, urine or saliva.

The prognosis and/or diagnosis of heart disease or heart failure includes the risk of developing HCM, HOCM, DCM or ISCM.

Preferably the method further comprises determining the methylation status and/or expression level at least one methylation marker selected from the group consisting of COX17 or MYBPC3.

Preferably the method further comprises determining the methylation status and/or expression level of at least one additional methylation marker selected from the group disclosed in Table 2.

In one embodiment the methylation status and/or expression level of the methylation of at least one of MSR1, HEY2, MFSD2B, MYBPC3 and/or PVT1 is determined.

This embodiment can be used in the prognosis and/or diagnosis of HCM or HOCM.

In another embodiment the methylation status and/or expression level of the methylation of at least one of TTPA, MYOM3, COX17, SMOC2, ITGBL1, and/or PVT1 is determined.

This embodiment can be used in the prognosis and/or diagnosis of ISCM.

In another embodiment the methylation status and/or expression level of the methylation of at least MRPL44, GALNT15, miR24-1, and/or PVT1 is determined.

This embodiment can be used in the prognosis and/or diagnosis of DCM.

The invention also provides a panel of biomarkers comprising at least one of the biomarkers selected from the group consisting of MFSD2B, MRPL44, TTPA, MYOM3, GALNT15, SMOC2, ITGBL1, MSR1, HEY2, miR24-1 and PVT1 in a plurality of biomarkers chosen from the list of biomarkers in Table 2.

Preferably the panel further comprises at least one methylation marker selected from the group consisting of COX17 and MYBPC3

The panel of biomarkers according to the invention can be used in the methods described herein.

The invention also provides the use of a biomarker selected from the group consisting of MSR1, HEY2, MFSD2B, MRPL44, TTPA, MYOM3, GALNT15, SMOC2, ITGBL1, miR24-1 and PVT1 for the prognosis and/or diagnosis of heart disease or heart failure.

The biomarkers of the invention can be used individually or preferably in a panel to assess

the risk of developing heart disease or heart failure, in particular HCM, HOCM, ISCM or DCM

the presence of heart disease or heart failure, in particular HCM, HOCM, ISCM or DCM, and/or

the progression of heart disease or heart failure, in particular HCM, HOCM, ISCM or DCM.

The invention therefore provides means for prognosing and/or diagnosing

the risk of developing heart disease or heart failure, in particular HCM, HOCM, ISCM or DCM

the presence of heart disease or heart failure, in particular HCM, HOCM, ISCM or DCM, and/or

the progression of heart disease or heart failure, in particular HCM, HOCM, ISCM or DCM, comprising

one or more means of detecting the methylation status and/or expression level of at least one methylation marker chosen from the group consisting of MSR1, HEY2, MFSD2B, MRPL44, TTPA, MYOM3, GALNT15, SMOC2, ITGBL1, miR24-1 and PVT1

The means can be presented in a kit.

The means or kit can be use for prognosing and/or diagnosing the risk of developing heart disease or heart failure in particular HCM, HOCM, ISCM or DCM.

The invention also provides a device for identifying heart disease or heart failure in a sample, in particular, HCM, HOCM, ISCM or DCM comprising:

(a) an analyzing unit comprising a detection agent for determining the methylation status and/or expression level of at least one methylation marker selected from the group consisting MSR1, HEY2, MFSD2B, MRPL44, TTPA, MYOM3, GALNT15, SMOC2, ITGBL1, miR24-1 and PVT1

(b) an evaluation unit comprising a data processor having tangibly embedded an algorithm for carrying out a comparison of the amount determined by the analyzing unit with a reference and which is capable of generating an output file containing a diagnosis established based on the said comparison.

### DETAILED DESCRIPTION OF THE INVENTION

The invention is described in further detail with reference to the following description and the figures.

The present invention provides and relates to novel methylation-sensitive protein-coding genes and non-coding RNA in patient subgroups and shows that methylation alterations are, in part, associated with alterations in corresponding gene/miRNA/IncRNA expression profiles.

The invention also provides and relates to the first comprehensive DNA methylation signature of cardiac tissue in HOCM patients which can be used to discover novel diagnostic and therapeutic targets for this incurable orphan disease.

The present inventors carried out a study of a novel cardiovascular-specific capture and performed targeted methylation sequencing of left ventricular tissue located at the interventricular septum (IVS) from a unique cohort of patients spanning 3 major HF etiologies—HOCM, DCM, and ISCM.

### BRIEF DESCRIPTION OF THE FIGURES

FIG. 1 shows DNA methylation of protein-coding genes and non-coding RNA that were significantly modulated in the studied HF patient cohort in A) Heatmap, B) Bar graph and C) Venn diagram illustrations.

FIG. 2 shows CpG methylation principal component analysis

### METHODS

Patients and Tissue Samples

The study population consisted of 39 male patients. Of these, 30 underwent cardiac surgery at the Cleveland Clinic, Ohio: 9 underwent orthotropic cardiac transplantation (OCT) for ISCM, 9 underwent OCT for DCM, and 12 underwent septal myectomy for HOCM. Another 9 patients represented an age- and gender-matched control group with non-failing hearts who died of non-cardiac causes. These patients donated hearts for OCT. The study conformed to the principles outlined in the Declaration of Helsinki. Ethical Approval for data collection and use of tissue was obtained from the Cleveland Clinic Institutional Review Board. Cardiac interventricular septal (IVS) tissue was surgically-removed, immediately snap frozen in liquid nitrogen, and stored at −80° C. until required for methylation profiling with no freeze-thaw cycles.

Methylation Sequencing from Left Ventricular Septal Tissue

Genomic DNA Isolation

Genomic DNA was isolated from 25 mg fresh-frozen IVS tissue derived from the left ventricle with the QIAamp DNA Mini Kit (Qiagen). DNA was eluted in 200 pl nuclease-free water and concentration was measured with Nanodrop. Quantification of double-stranded DNA was performed with Quant-iT PicoGreen dsDNA assay kit (Life Technologies) and fluorescence was measured with the Glomax Multi detection system (Promega) with excitation at 480 nm and emission at 520 nm.

DNA Library Preparation, Bisulfite Conversion, and Pre-Capture Library Amplification

One microgram of dsDNA in 50 pl nuclease free water was transferred into Covaris microTUBE AFA fiber screw-cap 6×16 tubes and sonicated into 250 bp long DNA fragments on Covaris M220 focused ultrasonicator. Sonication parameters were: time—120 sec, peak power—50.0, duty factor—20.0, cycles/burst—200. One microliter of fragmented DNA was used to assess the efficiency of sonication and fragment distribution with the Agilent High Sensitivity DNA Kit. The DNA chips were run on an Agilent 2100 Bioanalyser.

DNA samples that met the quality requirements were subsequently used for library construction. DNA Libraries were prepared from 1 μg fragmented dsDNA with the KAPA Library Preparation Kit, Illumina platforms (KAPA Biosystems, Boston, USA) according to the kit manual and as previously described¹. In brief, the process included: 1) End repair reaction followed by a SPRI bead cleanup; 2) A-tailing reaction and SPRI bead cleanup; 3) Adapter ligation (Roche NimbleGen SeqCap Adapter Kit A and B, final concentration of adapter: 1 μM) followed by two consecutive SPRI bead clean-ups; 4) Bisulfite conversion of adapter-ligated DNA libraries (EZ DNA Methylation Lightning Kit, Zymo Research); 5) Library amplification (SeqCap EZ Pre-Capture LM-PCR) with thermocycling parameters: 1 cycle (95° C.-2 min), 40 cycles (98° C.-30 sec, 60° C.-30 sec, 72° C.-4 min), 1 cycle (72° C.-10 min), 4° C.-Hold; and 6) Post-amplification cleanup with Agencourt Ampure XP beads (ratio of sample volume to beads is 1:1.8). Quantity and quality were assessed with the Quant-iT PicoGreen dsDNA assay and the Agilent High Sensitivity DNA Bioanalyser Assay.

Amplified Sample Library Quantification by Quantitative Real-Time Polymerase Chain Reaction (qRT-PCR)

Amplified bisulfite-converted DNA libraries were quantified using the KAPA Library Quantification Kit for Illumina Platforms. Samples were diluted 1/16 000 and reaction setup and cycling were performed according to the manufacturer protocol.

Amplified Sample Library Quality Control

Four nanomols from each quantified bisulfite-converted DNA library were suspended in 20 pl Elution buffer and used to assess library quality with the MiSeq Reagent Kit v3 (Illumina). Samples which met quality control criteria had a bisulfite conversion rate>98% and PCR duplicate rate<5%.

Custom Capture Design

The custom SeqCap Epi choice M probe pool (Roche Nimblegen, Madison, USA) was designed to include all known HF-related genes and ncRNA, as well as genes with known epigenetic regulation by DNA methylation. A list of 18582 putative promoter regions (−2000 and +500 bp from the transcriptional start site (TSS)) and enhancer regions of mRNA/miR/IncRNA and 17929 CpG islands was compiled following a comprehensive search of databases (NCBI Pubmed, LNCipedia, miRBase), published datasets (NCBI GEO (Gene expression Omnibus) public functional genomics data repository, NCBI GEO DataSets), and published articles (Pubmed) 2-10.

Library Hybridization to Custom Capture

One microgram sample library DNA was mixed with 10 pl bisulfite capture enhancer (SeqCap Epi Assessory kit), 1 pl (1000 pmol) SeqCap HE Universal Oligo (SeqCap HE Oligo kit), and 1 pl (1000 pmol) SeqCap HE Index oligo corresponding to the adapter. The mixture was air-dried in a vacuum concentrator at 60° C. for approximately 1.5 h. To each air-dried sample, 7.5 pl 2× Hybridization buffer and 2.5 pl Hybridization component A (SeqCap Hybridization and Wash Kit) were added. The mix was incubated at 95° C. for 10 min and added to 4.5 pl of the custom SeqCap Epi probe pool. Hybridization was performed by incubation for 64-72 h at 47° C.

Preparation of Captured Libraries for Methylation Sequencing

The captured DNA was washed and recovered with the use of the SeqCap Hybridization and Wash Kit and SeqCap Bead Capture kit as per kit instructions. Recovered captured DNA was amplified (SeqCap EZ Post-Capture LM-PCR) using the following thermocycling parameters: 1 cycle (98° C.-45 sec), 15 cycles (98° C.-15 sec, 60° C.-30 sec, 72° C.-30 sec), 1 cycle (72° C.-1 min), 4° C.-Hold. Post-amplification cleanup with Agencourt Ampure XP beads (ratio of sample volume to beads is 1:1.8) was performed as before. Quality and quantity were assessed, as above, with the High Sensitivity DNA Bioanalyser Assay and KAPA Library Quantification Kit, respectively. Next Generation Sequencing was performed on HiSeq 2500 platform with >180 m clusters per lane and 2×125 bp paired-end reactions at 60× at the Centre for Genomic Research at University of Liverpool (UK).

Sequence Data Pre-Processing, Alignment, and Post-Processing

Sequence data fastq files were checked for quality using FastQC (v0.11.5; https://www.bioinformatics.babraham.ac.uk/projects/fastqc/). Fastq files were then trimmed to remove poor quality bases (Phred score<20) and sequencing adapters using the BBDuk tool in the BBMap package (v35.14; http://jgi.doe.gov/data-and-tools/bbtools/bb-tools-user-guide/bbmap-guide/). The tools was run with trimq=20, qtrim=rl, k=31, mink=5, hdist=1, ktrim=r, and with tpe and tbo set as recommended. Trimmed fastq files were aligned to the hg19/GRCh37.75 human reference genome using BWA-meth (v0.10; ¹¹) under default settings. Bias plots were checked to ensure no deviation from the expected distribution of methylation across read positions, none of which was found. The output BAM file had duplicate sequences removed using MarkDuplicates in the PicardTools package (v1.105; https://broadinstitute.github.io/picard/).

The Bis-SNP package (v0.82.2¹²) was run according to the authors' standard protocol. Briefly, BisulfiteRealignerTargetCreator, BisulfitelndelRealigner and BisulfiteTableRecalibration were run, with BisulfiteCountCovariates before and after the recalibration step and diagnostic plots were checked to ensure Bis-SNP had performed as expected. The CalculateHsMetrics tools from PicardTools was run to determine total remaining reads and coverage. Finally, Bis-SNP BisulfiteGenotyper was used to produce a VCF format two callsets: one of CG methylated positions (run using the—C CG, 1), and one of single nucleotide variants (SNVs). These VCFs were subsequently postprocessed using Bis-SNPs VCFpostprocess. A version of this filtered VCF was converted to MethylKit¹³) input format for differential methylation analysis.

Differential Methylation Analysis

Analysis was run in the R Statistical Environment¹⁴ using MethylKit. Data was read in along with clinical information. Methylated positions per sample were filtered to those with at least 5× coverage. To determine differences between the different HF patient groups, each was compared to the NF control group. The sample set was normalized by the median and a principal component analysis (PCA) was conducted. This allowed an overview of both the clustering of patient samples into their respective subgroup as well as determining outliers based on distance from the relevant subgroup. For this we used the first two components of variance (PC1, PC2) because there was no obvious batch effect. Methylation profiles were then ‘tiled’ into 500 bp regions, and from these differential methylation was determined. Tiles with a false discovery rate (FDR) of >0.05, and with a difference in methylation of >10% were reported as being significantly differentially methylated.

NMF Clustering/Gene Network Analysis

Twelve samples (1 NF control, 5 HOCM, 4 DCM, 2 ISCM) were excluded from the non-negative matrix factorization (NMF) clustering analysis because more than 40% of the required methylation tile set for comparison was missing. A total of 62678 500 bp tiles without any missing values were extant at 5× coverage across the remaining 27 samples, reduced from a set of 133048 tiles. To determine the most divergent tiles, sets for each condition group with a mean difference of +/−15% from the control group were selected. NMF was conducted using the R ‘NMF’ package¹⁵ with k=5 based on the 4 conditions and one control group.

Ideogram generation was performed using Idiographica web-based software.

Assessment of Gene and Non-Coding RNA Expression in Methylation-Sensitive Regions Identified from Methylation Sequencing

RNA was extracted from 100 mg IVS tissue using the Trisure method (Bioline). The extracted RNA quality and concentration were determined with Nanodrop (Thermo Scientific).

mRNA

One microgram RNA was reverse transcribed to synthesize cDNA using SuperScript II reverse transcriptase (Invitrogen) and random primers (Invitrogen). Synthesized cDNA was diluted 1 in 5. Quantitative Real-Time Polymerase Chain Reaction (qRT-PCR) primers were designed for 28 genes with one primer spanning an exon/exon boundary to ensure amplification of only mature messenger RNA (mRNA). Primer sequences of a subset of 6 genes which expression was regulated by methylation included: COX17, F:ctcaggagaagaagccgct, R:cctttctcgatgatacacgca; CTGF, F:ggaagagaacattaagaagggc, R:ctccgggacagttgtaatgg; HEY2, F:tagagaaaaggcgtcgggat, R:gtgtgcgtcaaagtagcctt; MMP2, F:tgatcttgaccagaataccatcga, R:ggcttgcgagggaagaagtt; MSR1, F: ccaggtccaataggtcctcc, R:ctggccttccggcatatcc; MYOM3, F:aagtcctcgtccgcacttac, R:ggccaaacgtcgatcttttga. qRT-PCR was performed with Platinum SYBR Green qPCR SuperMix-UDG (Invitrogen) using the MX3005P System (Stratagene). The qRT-PCR cycling program consisted of 40 cycles of 15 seconds/95° C., 30 seconds/annealing temperature, and 30 seconds/72° C. Data were analyzed and relative expression determined using the comparative cycle threshold (Ct) method (2-ΔΔct), and expression was normalized to the housekeeper gene GAPDH, F: acagtcagccgcatcttctt, R: acgaccaaatccgttgactc.

Micro RNA

Fifty nanogram RNA was reverse transcribed to produce cDNA for TaqMan miRNA assays with the use of TaqMan MicroRNA Reverse Transcription Kit (Applied Biosystems) and miRNA-specific primers. TaqMan miRNA assays for: hsa-miR-155-5p (assay 002623), hsa-miR-23b-3p (assay 002126), hsa-miR-27b-3p (assay 002174), and hsa-miR-24-1-3p (assay 002440) were purchased from Applied Biosystems. TaqMan qRT-PCR was performed with TaqMan Fast Advanced Master Mix in triplicate on Quant Studio 7 Flex Real-time PCR System (Applied Biosystems). Each 20 pl reaction contained 4 pl cDNA, 10 pl Fast Advanced Master Mix, 1 pl TaqMan miR-specific primer, and 5 pl nuclease-free water. The qRT-PCR cycling program consisted of 1 cycle of 20 sec/95° C. and 40 cycles of 1 sec/95° C., 20 sec/60° C. Analysis was performed using the comparative Ct method and miRNA expression was normalized to expression of RNU48 control (assay 001006).

RNA Sequencing

In addition, total RNA and small RNA sequencing was carried out in the same samples to generate additional data on expression and differential methylation between heart failure sub-types and no heart failure controls. Sequencing was carried out using a Next Seq 500, and data was analysed with both Partek and CLC Genomics Workbench software.

Statistics

Statistical analysis of patient demographic and clinical data between all 4 patient groups was performed with the use of 1-way analysis of variance (ANOVA) or Kruskal-Wallis test for continuous variables for Gaussian or non-Gaussian data; or with Fisher exact test for categorical variables. For all other data, statistical analysis was performed between 2 patient groups: NF control group and one of HOCM, DCM, or ISCM groups. Unpaired t test or Mann-Whitney U test were used for Gaussian or non-Gaussian data, respectively. Statistical analysis was performed with GraphPad Prism V6.01.

Results

Clinical Classification of the Studied Patient Cohort

Characteristics of the studied patient cohort are listed in Table 1. There was no statistically-significant difference in age and body mass index between the groups.

TABLE 1
Patient Demographics and Clinical Characteristics

| | NF | HOCM | DCM | ISCM | |
|---|---|---|---|---|---|
| | n = 9 | n = 12 | n = 9 | n = 9 | P-value |
| Age (yrs) | 52 ± 7 | 51 ± 6 | 52 ± 4 | 53 ± 5 | 0.43 |
| BMI (kg/m²) | — | 30 [27.5-31.2] | 26.6 [25.8-33.7] | 27.5 [24.9-39.9] | 0.71 |
| Blood measurements | | | | | |
| CR (mg/dl) | — | 1.043 ± 0.13 | 1.278 ± 0.40 | 1.156 ± 0.27 | 0.17 |
| EGFR (ml/min) | — | 70 [66.3-70] | 57 [34-66.5] | 60 [56.4-63.5] | 0.002 |
| HB (g/dl) | — | 13.0 ± 2.5 | 12.8 ± 1.8 | 12.2 ± 1.7 | 0.69 |
| HCT (%) | — | 38.7 ± 7.7 | 39.0 ± 5.0 | 36.7 ± 4.2 | 0.68 |
| CHL (mg/dl) | — | 203.1 ± 37.8 | 141.3 ± 39.7 | 122.8 ± 28.3 | <0.0001 |
| LDL (mg/dl) | — | 121.1 ± 29.2 | 76.9 ± 34.9 | 62.2 ± 17.7 | <0.0001 |
| HDL (mg/dl) | — | 45.7 ± 8.2 | 36.4 ± 11.0 | 40.4 ± 23.8 | 0.11 |
| TG (ng/dl) | — | 181 ± 86 | 140 ± 91 | 100 ± 37 | 0.072 |
| TSH (U/ml) | — | 3.65 [2.37-5.40] | 3.03 [1.56-4.45] | 2.59 [1.48-10.60] | 0.71 |
| BNP (pg/ml) | — | 320 [101-510] | 671 [282-1000] | 516 [325-1695] | 0.17 |
| Medical history | | | | | |
| HTN (n, %) | 3 (33) | 4 (33) | 8 (89) | 6 (67) | 0.035 |
| DM (n, %) | — | 0 (0) | 2 (22) | 7 (78) | 0.002 |
| HLD (n, %) | — | 7 (58) | 7 (78) | 5 (56) | 0.76 |
| Smoker (n, %) | — | 4 (33) | 4 (44) | 6 (67) | 0.51 |
| Echocardiography | | | | | |
| LVEF (%) | 62 ± 7 | 62 ± 5 | 17 ± 8 | 14 ± 3 | <0.0001 |
| LVESD (cm) | — | 2.7 ± 0.4 | 5.9 ± 0.9 | 5.7 ± 1.3 | <0.0001 |
| LVEDD (cm) | — | 4.2 ± 0.3 | 6.7 ± 0.8 | 6.7 ± 1.2 | <0.0001 |
| RVSP (mmHg) | — | 29 ± 11 | 45 ± 12 | 47 ± 13 | 0.014 |

NF = normal function;
ISCM = Ischemic Cardiomyopathy;
HOCM = Hypertrophic Obstructive Cardiomyopathy;
DCM = Dilated Cardiomyopathy;
BMI = Body Mass Index;
CR = creatinine;
EGFR = Estimated Glomerular Filtration Rate;
HB = Haemoglobin;
HCT = Haematocrit;
CHL = Total Cholesterol;
LDL/HDL = Low/High-Density Lipoprotein;
TG = Triglycerides;
TSH = Thyroid-Stimulating Hormone;
BNP = B-type Natriuretic Peptide;
HTN = Hypertension;
DM = Diabetes Mellitus;
HDL = Hyperlipidemia;
LVEF = Left Ventricular Ejection Fraction;
LVESD/LVEDD = Left Ventricular End-Systolic/Diastolic Diameter;
RVSP = Right Ventricular Systolic Pressure.
Values are presented as mean ± SD, n (%), or median (interquartile range). Continuous variables were tested with the use of 1-way analysis of variance (ANOVA) or Kruskal-Wallis test. Categorical variables were tested with the use of Fisher exact test.

Altered DNA Methylation in HF Patients

A total of 62,678 500 bp-long differentially methylated regions (DMRs) were analyzed for altered methylation in interventricular septal tissue. A difference in methylation of 0% at 5× coverage with 5% FDR in each HF patient group when compared to the NF control group were considered for further analysis. We identified 195 unique DMRs in the HF cohorts versus control: 6 in HOCM, 151 in DCM, and 55 in ISCM patients.

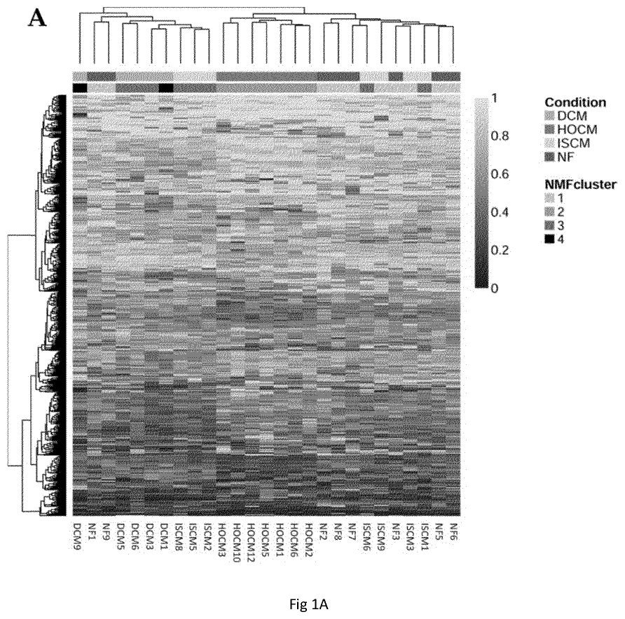

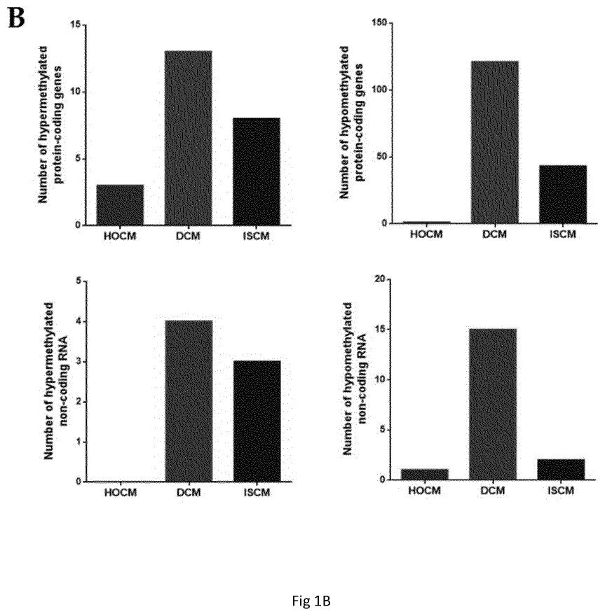

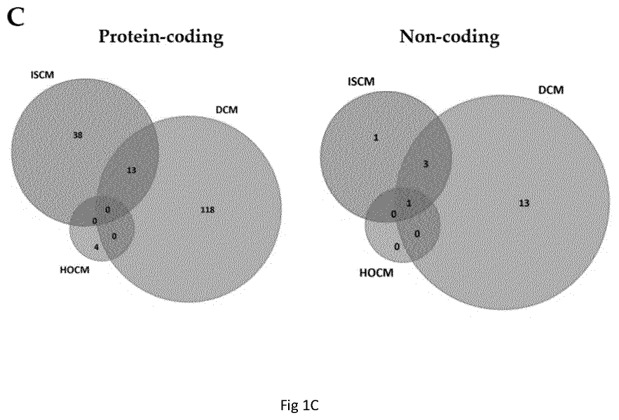

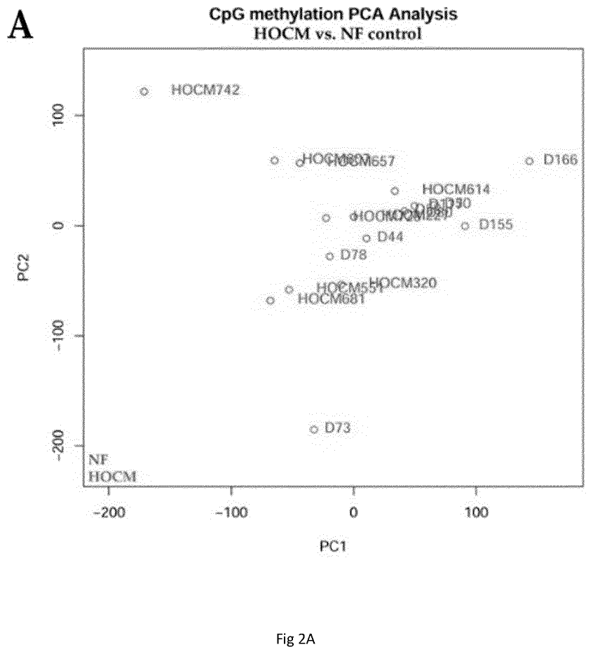

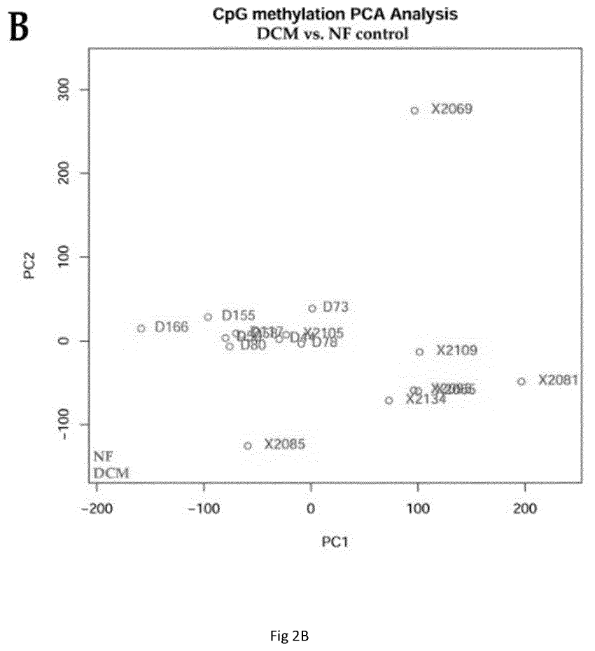

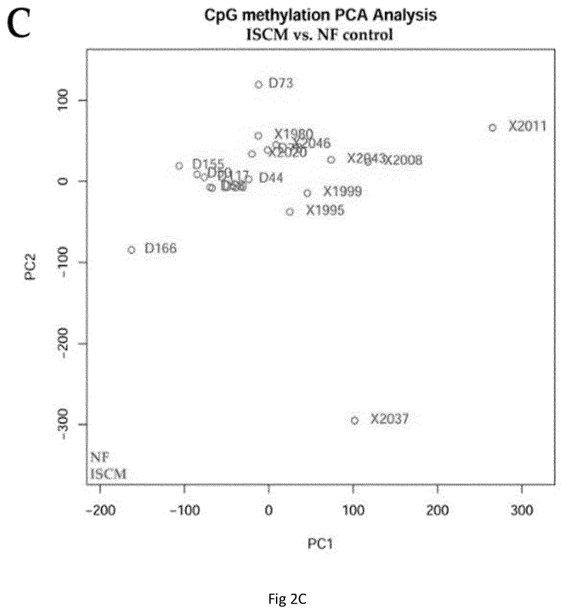

Non-negative matrix factorization (NMF) clustering (FIG. 1A) demonstrates subtle differences between HF subgroups. Such findings were expected considering that analyzed tissues were sourced from the left ventricular (LV) septum, and that the studied cohort consisted of HF patients who, despite differences in etiology, have common cardiac remodeling features. This is in contrast to other disease types such as cancer where big methylation differences are expected and evident. NMF clustering allowed a distinctive separation of the HOCM cohort, and to some degree in the DCM group, which had the greater number of identified DMRs. This was further supported by the PCA plots (FIG. 2) which indicated that patient samples from different HF disease groups are not highly divergent in the first two principal components but do cluster/separate as expected.

The identified regions were next annotated against known protein-coding genes and ncRNA and subdivided into regions with increased (hypermethylated) and reduced (hypomethylated) methylation (FIG. 1B). In the HOCM patient group, 5 protein-coding genes (4 hypermethylated, 1 hypomethylated) and 1 ncRNA (1 hypomethylated) were found to be differentially methylated. The DCM group was most divergent with 131 protein-coding genes (13 hypermethylated, 118 hypomethylated) and 17 ncRNA (3 hypermethylated, 14 hypomethylated) identified as having altered methylation profiles. In ISCM patients, 51 protein-coding genes (8 hypermethylated, 43 hypomethylated) and 5 ncRNA (3 hypermethylated, 2 hypomethylated) were differentially methylated. Venn diagrams were created to illustrate protein-coding genes and ncRNA which were methylated in patient group(s) (FIG. 1C).

### Detailed Description of the Figures

FIG. 1 DNA methylation of protein-coding genes and non-coding RNA that were significantly modulated in the studied HF patient cohort. A) Heatmap showing non-negative matrix factorization clustering of methylation profiles of NF Control, HOCM, DCM, and ISCM groups. The degree of methylation in each patient at n=690 500 bp tiles is presented from 0% (0, blue) to 100% (1, yellow). B) Bar graphs illustrating the number of hyper- and hypo-methylated protein-coding genes and non-coding RNA in HOCM, DCM, and ISCM groups as compared to the control, NF group. Differential hypomethylation of promoter regions is prominent in all 3 groups. C) Venn diagrams illustrating differential methylation profiles of HOCM, DCM, and ISCM as compared to NF control, in terms of the number of protein-coding genes (left) and non-coding RNA (miRNA and long non-coding RNA, right) involved. Methylation events specific to 1 and >1 patient group are shown. HOCM is depicted in purple colour, DCM—in green, ISCM—in blue.

FIG. 2 CpG methylation principal component analysis (PCA) plots showing the grouping/distribution of samples of each patient group (red spheres) versus the NF control group (blue spheres).

Aberrant DNA methylation regulates protein-coding gene and non-coding RNA expression in HF patients

To examine the impact of DNA methylation alterations at specific loci on gene expression, qRT-PCR analysis was performed. Total RNA and small RNA sequencing was also conducted to examine methylation changes and impact on expression at a genomic level. qRT-PCR and RNA sequencing was performed for all 39 patients.

TABLE 2
Significant differential methylation levels of protein-coding genes and
non-coding RNAs in Heart Failure patient groups versus NF controls

| | | Patient | % | |
|---|---|---|---|---|
| | | group | Methylation | |
| | | where | difference | |
| | | significant | vs. NF | |
| Gene/miR/ | Direction of | methylation | control | |
| lncRNA | methylation | identified | group | P-FDR |
| HEY2 | hypermethylated | HOCM | 15.81 | 0.006 |
| MSR1 | hypermethylated | HOCM | 19.87 | 0.044 |
| MFSD2B | hypermethylated | HOCM | 21.64 | 0.005 |
| MYBPC3 | Hypermethylated | HOCM | 10.12 | 0.048 |
| TTPA | hypermethylated | ISCM | 19.44 | 0.0000001 |
| COX17 | hypermethylated | ISCM | 25.99 | 0.048 |
| MYOM3 | hypermethylated | ISCM | 21.25 | 0.003 |
| KRT5 | hypermethylated | ISCM | 15.20 | 0.041 |
| | | DCM | 16.84 | 0.007 |
| TBX2 | hypermethylated | DCM | 17.48 | 0.013 |
| MRPL44 | hypermethylated | DCM | 16.18 | 0.024 |
| BRAF | hypermethylated | DCM | 13.56 | 0.039 |
| GALNT15 | hypermethylated | DCM | 13.56 | 0.008 |
| miR23b, | hypermethylated | ISCM | 11.27 | 0.038 |
| miR27b, | | DCM | 15.08 | 0.003 |
| miR24-1 | | | | |
| MUC5B | hypomethylated | HOCM | 18.17 | 0.010 |
| PAIP1 | hypomethylated | ISCM | 20.89 | 0.048 |
| PXDN | hypomethylated | ISCM | 11.46 | 0.032 |
| TGFB1 | hypomethylated | ISCM | 12.37 | 0.002 |
| SMOC2 | hypomethylated | ISCM | 16.33 | 0.027 |
| ITGBL1 | hypomethylated | ISCM | 10.51 | 0.014 |
| C1QTNF7 | hypomethylated | ISCM | 10.50 | 0.032 |
| CYR61 | hypomethylated | ISCM | 13.03 | 0.032 |
| | | DCM | 14.10 | 0.008 |
| ACSL1 | hypomethylated | ISCM | 17.50 | 0.00001 |
| | | DCM | 11.88 | 0.007 |
| CTGF | hypomethylated | ISCM | 17.52 | 0.00003 |
| | | DCM | 11.42 | 0.019 |
| HMOX1 | hypomethylated | ISCM | 22.55 | 0.041 |
| COL3A1 | hypomethylated | DCM | 10.60 | 0.039 |
| KDM5B | hypomethylated | DCM | 11.18 | 0.028 |
| DENND5A | hypomethylated | DCM | 11.21 | 0.009 |
| SMAD2 | hypomethylated | DCM | 13.05 | 0.030 |
| COL19A1 | hypomethylated | DCM | 13.47 | 0.031 |
| MMP2 | hypomethylated | DCM | 14.45 | 0.033 |
| WNT11 | hypomethylated | DCM | 15.61 | 0.007 |
| FBLN2 | hypomethylated | DCM | 18.21 | 0.011 |
| SHB | hypomethylated | DCM | 10.79 | 0.037 |
| MN1 | hypomethylated | DCM | 10.79 | 0.027 |
| SCUBE2 | hypomethylated | DCM | 12.05 | 0.039 |
| PDE4C | hypomethylated | DCM | 12.20 | 0.011 |
| RASSF9 | hypomethylated | DCM | 13.95 | 0.008 |
| CYS1 | hypomethylated | DCM | 14.30 | 0.008 |
| miR155 | hypomethylated | ISCM | 16.41 | 0.006 |
| miR21 | hypomethylated | DCM | 10.39 | 0.046 |
| miR23b, | hypomethylated | DCM | 10.43 | 0.032 |
| miR27b | | | | |
| PVT1 | hypomethylated | HOCM | 12.68 | 0.049 |
| | | ISCM | 11.20 | 0.003 |
| | | DCM | 16.11 | 0.009 |
| | | DCM | 20.18 | 0.016 |

P-FDR = False Discovery Rate (FDR)-adjusted p-value;
miR = micro RNA;
lncRNA = long non-coding RNA

In silico analysis of the specific methylated regions identified in the putative promoters (−2000/+500 bp from the transcriptional start site) of these coding/non-coding RNA revealed that these sites contain active transcription marks including H3K27ac (UCSC genome browser, hg19). This supports the fact that the methylation alterations at such potential regulatory regions could plausibly impact gene expression across the various sample types.

Table 3 highlights differentially methylated protein coding genes and non-coding RNAs with associated significant changes in expression levels. The patterns of gene expression were consistent with the direction of DNA methylation, i.e. genes with hypermethylated promoters incurred reduced gene expression compared to the NF group, whereas those with hypomethylated promoters had increased gene levels. In addition, MYBPC3 had differential gene hypermethylation in heart failure, including HOCM, versus control, even at the single base pair resolution.

Examples of such expression changes in Table 3 are as follows; HEY2 and MSR1 were significantly hypermethylated in HOCM (15.81%, p=0.006 and 19.87%, p=0.044) with gene expression significantly reduced by 0.53-fold (p=0.001) and 0.42-fold (p=0.003), respectively, in HOCM versus the NF control group. MYOM3 and COX17 were hypermethylated in ISCM (21.25%, p=0.003 and 25.99%, p=0.046), and their transcript levels were significantly reduced by 0.74-fold (p=0.019) and 0.49-fold (p=0.001), respectively. As examples of hypomethylated genes, MMP2 was significantly hypomethylated in DCM (14.45%, p=0.032), and CTGF—in ISCM (17.52%, p=0.00003) and DCM (11.42%, p=0.019) at two neighboring DMR (Table 1). Expression levels of MMP2 were increased by 2.67-fold in DCM (p=0.003), and CTGF was upregulated by 2.85-fold in ISCM (p=0.005) and 3.33-fold in DCM (p=0.011).

From a ncRNA perspective, DNA methylation analysis showed the miR-23b/miR-27b/miR24-1 cluster to be significantly hypermethylated in ISCM (11.27%, p=0.035) and DCM (15.08%, p=0.003) at two different regions, and miR-155 to be hypomethylated in ISCM (16.41%, p=0.005). Differential expression was also detected.

TABLE 3
Methylation and expression levels of selected protein-coding genes, miRNAs, and long
non-coding RNA linked to methylated DMR in HF patient groups versus NF controls

| | | | | | Fold | |
|---|---|---|---|---|---|---|
| | | | | | gene/ | |
| | | | % | | miRNA | |
| | | HF patient | Methylation | | express | |
| | | group where | difference | | ion vs. | |
| | | significant | vs. NF | | NF | |
| Gene/ | Direction of | methylation | control | | control | |
| miRNA | methylation | identified | group | P-FDR | group | P-value |
| HEY2 | hypermethylated | HOCM | 15.81 | 0.006 | 0.53 | 0.001 |
| MSR1 | hypermethylated | HOCM | 19.87 | 0.044 | 0.42 | 0.003 |
| COX17 | hypermethylated | ISCM | 25.99 | 0.046 | 0.49 | 0.001 |
| MYOM3 | hypermethylated | ISCM | 21.25 | 0.003 | 0.74 | 0.019 |
| GALNT15 | hypermethylated | DCM | 13.46 | 0.008 | 0.19 | 0.001 |
| miR24-1^{§} | hypermethylated | ISCM | 11.27 | 0.035 | 0.81 | 0.031 |
| CTGF | hypomethylated | ISCM | 17.52 | 0.00003 | 2.85 | 0.005 |
| | hypomethylated | DCM | 11.42 | 0.019 | 3.33 | 0.011 |
| MMP2 | hypomethylated | DCM | 14.45 | 0.032 | 2.67 | 0.003 |
| ITGBL1 | hypomethylated | ISCM | 10.51 | 0.014 | 2.20 | 0.001 |
| SMOC2 | hypomethylated | ISCM | 16.33 | 0.027 | 3.45 | 0.001 |
| miR155 | hypomethylated | ISCM | 16.41 | 0.005 | 1.63 | 0.030 |

p-FDR, False Discovery Rate corrected p-value;
DMR, differentially methylated region;
^{§}miR24-1 hypermethylation is identified as part of the miR23b/miR27b/miR24-1 cluster

### REFERENCES

1. Ponikowski P, Voors A A, Anker S D, Bueno H, Cleland J G, Coats A J, Falk V, Gonzalez-Juanatey J R, Harjola V P, Jankowska E A, Jessup M, Linde C, Nihoyannopoulos P, Parissis J T, Pieske B, Riley J P, Rosano G M, Ruilope L M, Ruschitzka F, Rutten F H, van der Meer P, Authors/Task Force M and Document R. 2016 ESC Guidelines for the diagnosis and treatment of acute and chronic heart failure: The Task Force for the diagnosis and treatment of acute and chronic heart failure of the European Society of Cardiology (ESC). Developed with the special contribution of the Heart Failure Association (HFA) of the ESC. Eur J Heart Fail. 2016; 18:891-975.
2. Writing Group M, Mozaffarian D, Benjamin E J, Go A S, Arnett D K, Blaha M J, Cushman M, Das S R, de Ferranti S, Despres J P, Fullerton H J, Howard V J, Huffman M D, Isasi C R, Jimenez M C, Judd S E, Kissela B M, Lichtman J H, Lisabeth L D, Liu S, Mackey R H, Magid D J, McGuire D K, Mohler E R, 3rd, Moy C S, Muntner P, Mussolino M E, Nasir K, Neumar R W, Nichol G, Palaniappan L, Pandey D K, Reeves M J, Rodriguez C J, Rosamond W, Sorlie P D, Stein J, Towfighi A, Turan T N, Virani S S, Woo D, Yeh R W, Turner M B, American Heart Association Statistics C and Stroke Statistics S. Heart Disease and Stroke Statistics-2016 Update: A Report From the American Heart Association. Circulation. 2016; 133:e38-360.
3. Heidenreich P A, Albert N M, Allen L A, Bluemke D A, Butler J, Fonarow G C, Ikonomidis J S, Khavjou O, Konstam M A, Maddox T M, Nichol G, Pham M, Pina I L and Trogdon J G. Forecasting the impact of heart failure in the United States: a policy statement from the American Heart Association. Circulation Heart failure. 2013; 6:606-19.
4. Papait R, Greco C, Kunderfranco P, Latronico M V and Condorelli G. Epigenetics: a new mechanism of regulation of heart failure? Basic Res Cardiol. 2013; 108:361.
5. Di Salvo T G and Haldar S M. Epigenetic mechanisms in heart failure pathogenesis. Circulation Heart failure. 2014; 7:850-863.
6. Greco C M and Condorelli G. Epigenetic modifications and noncoding RNAs in cardiac hypertrophy and failure. Nature reviews Cardiology. 2015; 12:488-97.
7. Kim S Y, Morales C R, Gillette T G and Hill J A. Epigenetic regulation in heart failure. Current opinion in cardiology. 2016; 31:255-65.
8. Jaenisch R and Bird A. Epigenetic regulation of gene expression: how the genome integrates intrinsic and environmental signals. Nat Genet. 2003; 33 Suppl:245-54.
9. Russell-Hallinan A, Watson C J and Baugh J. Epigenetics of Aberrant Cardiac Wound Healing. Comprehensive Physiology. 2018; in press.
10. Movassagh M, Choy M K, Knowles D A, Cordeddu L, Haider S, Down T, Siggens L, Vujic A, Simeoni I, Penkett C, Goddard M, Lio P, Bennett M R and Foo R S. Distinct epigenomic features in end-stage failing human hearts. Circulation. 2011; 124:2411-22.
11. Haas J, Frese K S, Park Y J, Keller A, Vogel B, Lindroth A M, Weichenhan D, Franke J, Fischer S, Bauer A, Marquart S, Sedaghat-Hamedani F, Kayvanpour E, Kohler D, Wolf N M, Hassel S, Nietsch R, Wieland T, Ehlermann P, Schultz J H, Dosch A, Mereles D, Hardt S, Backs J, Hoheisel J D, Plass C, Katus H A and Meder B. Alterations in cardiac DNA methylation in human dilated cardiomyopathy. EMBO molecular medicine. 2013; 5:413-29.
12. Meder B, Haas J, Sedaghat-Hamedani F, Kayvanpour E, Frese K, Lai A, Nietsch R, Scheiner C, Mester S, Bordalo D M, Amr A, Dietrich C, Pils D, Siede D, Hund H, Bauer A, Holzer D B, Ruhparwar A, Mueller-Hennessen M, Weichenhan D, Plass C, Weis T, Backs J, Wuerstle M, Keller A, Katus H A and Posch A E. Epigenome-Wide Association Study Identifies Cardiac Gene Patterning and a Novel Class of Biomarkers for Heart Failure. Circulation. 2017; 136:1528-1544.
13. Jo B S, Koh I U, Bae J B, Yu H Y, Jeon E S, Lee H Y, Kim J J, Choi M and Choi S S. Methylome analysis reveals alterations in DNA methylation in the regulatory regions of left ventricle development genes in human dilated cardiomyopathy. Genomics. 2016; 108:84-92.
14. Koczor C A, Lee E K, Torres R A, Boyd A, Vega J D, Uppal K, Yuan F, Fields E J, Samarel A M and Lewis W. Detection of differentially methylated gene promoters in failing and nonfailing human left ventricle myocardium using computation analysis. Physiol Genomics. 2013; 45:597-605.
15. Gil-Cayuela C, Rosello L E, Tarazon E, Ortega A, Sandoval J, Martinez-Dolz L, Cinca J, Jorge E, Gonzalez-Juanatey J R, Lago F, Rivera M and Portoles M. Thyroid hormone biosynthesis machinery is altered in the ischemic myocardium: An epigenomic study. Int J Cardiol. 2017; 243:27-33.
16. Li B, Feng Z H, Sun H, Zhao Z H, Yang S B and Yang P. The blood genome-wide DNA methylation analysis reveals novel epigenetic changes in human heart failure. Eur Rev Med Pharmacol Sci. 2017; 21:1828-1836.

1. A method of prognosing and/or diagnosing heart disease or heart failure in a subject, comprising
determining the methylation status and/or expression level of at least one methylation marker selected from the group consisting of
MFSD2B, miR24-1, TTPA, GALNT15, ITGBL1, SMOC2, MSR1, PVT1, MYOM3, HEY2 and MRPL44; and/or
determining the methylation status and/or expression level of at least one methylation marker selected from the group consisting of COX17 and MYBPC3,
wherein the methylation status and/or expression level of at least one methylation marker is indicative of the prognosis and/or diagnosis of said subject.

2. (canceled)

3. A method as claimed in claim 1 carried out on a sample from the subject.

4. A method as claimed in claim 3 wherein the sample is chosen from blood, cardiac tissue, urine or saliva.

5. The method of claim 1, wherein the prognosis and/or diagnosis of heart disease or heart failure includes the risk of developing HCM, HOCM, DCM or ISCM.

6. The method of claim 1, wherein
the method comprises determining the methylation status and/or expression level of at least one methylation marker selected from the group consisting of
MFSD2B, miR24-1, TTPA, GALNT15, ITGBL1, SMOC2, MSR1, PVT1, MYOM3, HEY2 and MRPL44; and
further comprises determining the methylation status and/or expression level at least one methylation marker selected from the group consisting of COX17 and MYBPC3.

7. The method of claim 1, wherein the method further comprises determining the methylation status and/or expression level of at least one additional methylation marker selected from the group disclosed in Table 2.

8. The method of claim 1, wherein the methylation status and/or expression level of the methylation of at least one of MSR1, HEY2, MFSD2B, MYBPC3 and/or PVT1 is determined.

9. The method of claim 1, wherein the prognosis and/or diagnosis of heart disease or heart failure includes the risk of developing HCM or HOCM.

10. The method of claim 1, wherein the methylation status and/or expression level of the methylation of at least one of TTPA, MYOM3, COX17, SMOC2, ITGBL1 and/or PVT1 is determined.

11. The method of claim 1, wherein the prognosis and/or diagnosis of heart disease or heart failure includes the risk of developing ISCM.

12. The method of claim 1, wherein the methylation status and/or expression level of the methylation of at least MRPL44, GALNT15, miR24-1 and/or PVT1 is determined.

13. The method of claim 1, wherein the prognosis and/or diagnosis of heart disease or heart failure includes the risk of developing DCM.

14. A panel of biomarkers comprising at least one of the biomarkers selected from the group consisting of MFSD2B, MRPL44, TTPA, MYOM3, GALNT15, SMOC2, ITGBL1, MSR1, HEY2, miR24-1 and PVT1 in a plurality of biomarkers chosen from the list of biomarkers in Table 2 for use in a method as claimed in claim 1.

15. (canceled)

16. The panel of biomarkers of claim 14, for use in a method to assess
the risk of developing heart disease or heart failure, in particular HCM, HOCM, ISCM or DCM
the presence of heart disease or heart failure, in particular HCM, HOCM, ISCM or DCM, and/or
the progression of heart disease or heart failure, in particular HCM, HOCM, ISCM or DCM.

17. A kit for prognosing and/or diagnosing
the risk of developing heart disease or heart failure, in particular HCM, HOCM, ISCM or DCM
the presence of heart disease or heart failure, in particular HCM, HOCM, ISCM or DCM, and/or
the progression of heart disease or heart failure, in particular HCM, HOCM, ISCM or DCM, comprising
one or more means of detecting the methylation status and/or expression level of at least one methylation marker chosen from the group consisting of MFSD2B, MRPL44, TTPA, MYOM3, GALNT15, SMOC2, ITGBL1, MSR1, HEY2, miR24-1 and PVT1.

18. Use of the kit of claim 17 for prognosing and/or diagnosing the risk of developing heart disease or heart failure in particular HCM, HOCM, ISCM or DCM.

19. A device for identifying heart disease or heart failure in a sample, in particular, HCM, HOCM, ISCM or DCM comprising:
(a) an analyzing unit comprising a detection agent for determining the methylation status and/or expression level of at least one methylation marker selected from the group consisting of MFSD2B, MRPL44, TTPA, MYOM3, GALNT15, SMOC2, ITGBL1, MSR1, HEY2, miR24-1 and PVT1
(b) an evaluation unit comprising a data processor having tangibly embedded an algorithm for carrying out a comparison of the amount determined by the analyzing unit with a reference and which is capable of generating an output file containing a diagnosis established based on the said comparison.
